(19) United States
(12) Patent Application Publication
(10) Pub. No.: US 2009/0259036 A1
(43) Pub. Date: Oct. 15, 2009

(54) EXTRACTION OF LESS POLAR IMPURITIES FROM SUCRALOSE CONTAINING AQUEOUS FEED STREAMS

(75) Inventor: James Edwin Wiley, JR., Daphne, AL (US)

Correspondence Address: Ratner Prestia, P.O. Box 980, Valley Forge, PA, 19482, US

(73) Assignee: Tate & Lyle technology Limited, London (GB)

(21) Appl. No.: 12/416,541

(22) Filed: Apr. 1, 2009

Related U.S. Application Data
(60) Provisional application No. 61/042,076, filed on Apr. 3, 2008.

(51) Int. Cl. C07H 1/06

(52) U.S. Cl. 536/127

(57) ABSTRACT

A process for the purification of sucralose containing aqueous feed streams is disclosed. The process comprises the step of extracting an aqueous feed stream comprising sucralose and impurities less polar than sucralose, such as tetrachloro saccharides, with an organic solvent that is immiscible with water, such as ethyl acetate. In this step, the mass ratio of organic solvent to aqueous feed stream is in the range of 0.4 to 0.9. Greater than 50% of the sucralose and greater than 95% of the tetrachloro saccharide impurities are extracted into the organic extract. The organic extract is back extracted with water and the resulting aqueous extract recycled to the initial extraction step.

### CROSS REFERENCE TO RELATED APPLICATIONS

This application claims priority benefit of U.S. Provisional Appln. No. 61/042,076, filed Apr. 3, 2008, the entirety of which is incorporated herein by reference.

### FIELD OF THE INVENTION

This invention relates to sucralose and to methods for its preparation. In particular, this invention relates to the extraction of impurities less polar than sucralose from sucralose containing aqueous feed streams.

### BACKGROUND OF THE INVENTION

Sucralose (4,1′,6′-trichloro-4,1′,6′-trideoxygalactosucrose), a high-intensity sweetener that can be used in many food and beverage applications, is a galacto-sucrose having the following molecular structure:

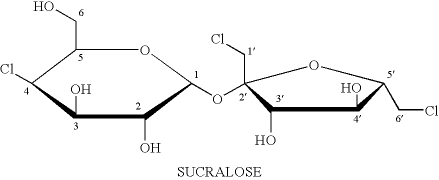

Sucralose is made from sucrose by converting the hydroxyls in the 4, 1′, and 6′ positions to chloro groups. In this process, the stereochemical configuration at the 4 position is inverted.

In one process for making sucralose from sucrose, sucrose is first converted to a sucrose-6-ester, such as sucrose-6-acetate or sucrose-6-benzoate. The sucrose-6-ester is chlorinated by reaction with a chlorination agent and a tertiary amide, and the resulting reaction mixture heated and then quenched with aqueous alkali. The resulting 4,1′,6′-trichloro-4,1′,6′-trideoxygalactosucrose ester (sucralose-6-ester) is converted to sucralose, which is subsequently purified and isolated.

This process typically provides a product that contains varying amounts of other chlorinated sugar compounds in addition to sucralose. During removal of these impurities the loss of sucralose should be minimized, and the purification and isolation process should be economical to operate on a large scale. Although advances have been made in the purification of sucralose, there is a continuing need for processes that remove impurities from sucralose, produce sucralose in high purity, minimize the yield loss in the purification process, and are economical to operate on a large scale.

### SUMMARY OF THE INVENTION

In one aspect, the invention is a process for the purification of sucralose containing feed streams, the process comprising the steps of:
a) extracting an aqueous stream comprising sucralose and tetrachloro saccharides with an organic solvent and producing a first organic extract and a first aqueous extract, in which the organic solvent is immiscible with water, and in which greater than 50% of the sucralose and at least 95% of the tetrachloro saccharides in the aqueous stream pass into the first organic extract;
b) extracting the first organic extract with an aqueous solvent to produce a second organic extract and a second aqueous extract; in which the sucralose preferentially passes into the second aqueous extract; and
c) adding the second aqueous extract to step a).

In one aspect of the invention, the organic solvent is ethyl acetate. In one aspect of the invention, the mass ratio of organic solvent to aqueous feed stream in step a) is about 0.4 to about 0.9. In one aspect of the invention, in step b) greater than 90% of the sucralose in the first organic extract is extracted into the second aqueous extract.

### BRIEF DESCRIPTION OF THE DRAWINGS

FIG. 1 is a flow diagram showing the process of the invention.

FIG. 2 shows the effect of the ratio of organic solvent (“solvent”) to aqueous sucralose containing feed stream (“feed”) at constant sucralose yield on the purity of the sucralose in the first aqueous extract.

### DETAILED DESCRIPTION OF THE INVENTION

Unless the context indicates otherwise, in the specification and claims, the terms organic solvent, tetrachloro saccharide, trichloro saccharide, dichloro saccharide, salt, sucralose-6-ester, carbohydrate, and similar terms also include mixtures of such materials. The term saccharide includes monosaccharide, disaccharides, and polysaccharides. Solvent means a liquid that dissolves another material. An aqueous solvent is one in which water is the primary (greater than 50 vol % of the solvents present) or only solvent. Two solvents are immiscible if, in any proportion, they do not form a homogeneous phase. Unless otherwise specified, all percentages are percentages by weight and all solvent ratios are volume to volume.

A process for the preparation of sucralose from sucrose involves the following steps. First, the hydroxyl in the 6 position of sucrose is blocked with an ester group, such as acetate or benzoate. Then the hydroxyls in the 4, 1′, and 6′ positions of the resulting sucrose 6-ester are converted to chloro groups, with inversion of the stereochemical configuration at the 4 position. Conversion of the hydroxyls in the 4, 1′, and 6′ positions of the ester to chloro groups with inversion of the stereochemical configuration at the 4 position is disclosed in Walkup, U.S. Pat. No. 4,980,463; Jai, U.S. Pat. Pub. 2006/0205936 A1; and Fry, U.S. Pat. Pub. 2007/0100139 A1; the disclosures of which are all incorporated herein by reference. Then the ester group in the 6 position of the resulting sucralose-6-ester is removed, and sucralose, the resulting product, purified and isolated. The process, or any of the individual steps thereof, can be either batch or continuous processes.

### Purification of Sucralose Containing Feed Streams

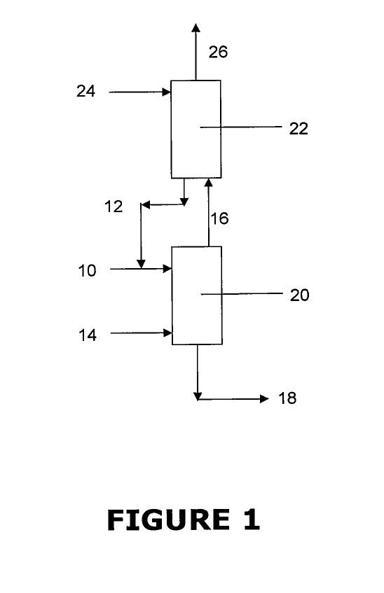

Referring to FIG. 1, following conversion of sucralose-6-ester to sucralose, an aqueous feed stream (10) that comprises sucralose is produced. Aqueous feed stream 10 typically comprises a total of about of 6 wt % to 50 wt %, for example, about 6 wt % to 12 wt %, about 12 wt % to 18 wt %, about 18 wt % to 25 wt %, or about 25 wt % to about 50 wt % of carbohydrates in a stream in which water is the primary or only solvent. Of the carbohydrates present, between 50% and 80% are typically sucralose. The other carbohydrates primarily fall into one of three categories based on the number of chlorine atoms on the molecule: tetrachloro saccharide impurities (tetrachloro saccharides), dichloro saccharide impurities (dichloro saccharides), and trichloro saccharide impurities (trichloro saccharides). The location and extent of chlorination strongly affects the polarity of the resulting saccharide. In general, the tetrachloro saccharides impurities are less polar than sucralose, and the dichloro saccharide impurities are more polar than sucralose. Generally, more polar impurities are more soluble than sucralose in more polar solvents, and less polar impurities are more soluble than sucralose in less polar solvents.

Other materials that can be present in aqueous feed stream 10 include inorganic salts, such as alkali metal chlorides such as sodium chloride, alkaline earth chlorides, and ammonium chloride; and organic salts, primarily alkali metal acetates, such as sodium acetate; dimethyl amine hydrochloride; and alkali metal formates, such as sodium formate. A small amount, typically less than 5,000 ppm, of the polar aprotic solvent used in the chlorination step, typically N,N-dimethyl formamide, can also be present in the feed stream.

Aqueous feed stream 10 and second aqueous extract 12, discussed below, are combined to produce a combined aqueous stream, which is extracted with a stream of an organic solvent (14) to produce a first organic extract (16) and a first aqueous extract (18). This extraction step is referred to as step EXT1. Because the less polar compounds are preferentially extracted into first organic extract 16, this extraction removes less polar compounds, which include the tetrachloro saccharides, from the combined aqueous stream. The extraction is carried out under conditions in which greater than 50%, greater than 55%, greater than 60%, or greater than 65%, of the sucralose and 95% of the tetrachloro saccharide impurities in the aqueous feed stream are extracted into first organic extract 16.

The choice of solvent is determined by the relative solubilities of sucralose and the principal impurities in the organic solvent and in the aqueous feed stream, as well as such other factors as flammability, ease of recycling within the process, environmental concerns, toxicity, and cost. The organic solvent can be intentionally saturated with water before use in the extraction step. Mixtures of organic solvents can be used. Solvents contemplated for use as the organic solvent include those that are immiscible with water and in which halogenated sucrose derivatives, such as sucralose, are readily soluble. Also included are solvents that are partially soluble in a first solvent such as water, an aqueous solution, or other solvent in which halogenated sucrose derivatives are readily soluble, but in which the second solvent still forms a separate phase when mixed with the first solvent in proper ratios and under proper conditions. Typical organic solvents include, but are not limited to, methyl acetate, ethyl acetate, methyl ethyl ketone, methyl iso-butyl ketone, methyl iso-amyl ketone, methylene chloride, chloroform, diethyl ether, methyl t-butyl ether, n-pentane, n-hexane, n-heptane, n-octane, isooctane, 1,1,1-trichloroethane, n-dodecane, white spirit, turpentine, cyclohexane, propyl acetate, butyl acetate, amyl acetate, carbon tetrachloride, xylene, toluene, benzene, trichloroethylene, 2-butoxyethanol acetate (butyl CELLOSOLVE® acetate), ethylene dichloride, butanol, morpholine, and mixtures thereof. The first organic solvent preferably comprises methyl acetate, ethyl acetate, iso-propyl acetate, n-propyl acetate, n-butyl acetate, amyl acetate, methyl ethyl ketone, methyl iso-butyl ketone, methyl iso-amyl ketone, methylene chloride, chloroform, or n-butanol, either as a single solvent, or as a mixed solvent with these solvents, or with other solvents from the first list. The first solvent more preferably comprises ethyl acetate, iso-propyl acetate, n-propyl acetate, n-butyl acetate, methyl iso-butyl ketone, or n-butanol, either as a single solvent, or as a mixed solvent with these solvents, or with other solvents from the first or second list. Ethyl acetate is the most preferred solvent. Diethyl ether, methyl t-butyl ether, n-pentane, n-hexane, n-heptane, n-octane, isooctane, 1,1,1-trichloroethane, n-dodecane, white spirit, turpentine, cyclohexane, carbon tetrachloride, xylene, toluene, benzene, trichloroethylene, 2-butoxyethanol acetate (butyl CELLOSOLVE® acetate), ethylene dichloride, and morpholine are generally not preferred as single solvents, but may be used in mixed solvents as described.

Extraction is carried out in a first liquid extractor (20), which can be any type of liquid-liquid extractor known in the art, for example, a conventional mixer-settler or a bank of conventional mixer-settlers, an Oldshue-Rushton multiple-mixer column, a sieve tray column, a random packed column, a pulsed packed column, a structured (SMVP) packing column, an asymmetric rotating disk extractor (ARD), a KARR® column, a Kuhni extractor, a Treybel extractor, a Scheibel column, a rotating disc contactor (RDC) column, or a centrifugal extractor such as a Podbielniak centrifugal extractor or a Robatel centrifugal extractor. An extractor with five or more theoretical stages of extraction can be used. A stream of organic solvent (14), which if desired can be saturated with water, for example ethyl acetate saturated with water, is fed to the bottom of extractor 20 in proportion to the total amount of feed to the top of extractor 20.

First aqueous extract 18 comprises sucralose as well as some impurities, primarily salts and saccharide impurities that are more polar than sucralose or which have about the same polarity as sucralose. Sucralose can be isolated from first aqueous extract 18 by concentrating the extract by evaporating the water and then isolating the sucralose by crystallization. However, alternatively, first aqueous extract 18 can be used as the feed stream for additional purification steps.

First organic extract 16 is sent to a second liquid extractor (22) to recover sucralose from first organic extract 16 while leaving the bulk of the less polar impurities in an organic extract. This extraction step is referred to as step EXT1B. If the process comprises additional purification steps, if desired, one or more other recycle streams from these additional purification steps can be recycled to the second liquid extractor 22. Second liquid extractor 22 can be any type of liquid-liquid extractor known in the art, examples of which are listed above. An extractor with five or more theoretical stages of extraction can be used. First organic extract 16 is fed into the bottom of liquid extractor 22. A stream (24) of water, which if desired can be saturated with the same organic solvent used in first liquid extractor 20, for example water saturated with ethyl acetate, is fed into the top of extractor 22. The mass ratio of water to first organic extract 16 is typically about 0.8 to about 0.9. An interface between the two phases is maintained in the bottom of second liquid extractor 22 where the aqueous phase, second aqueous extract 12, is collected. Second aqueous extract 12 is recycled to first liquid extractor 20. Greater than 85%, 90%, 92%, or 95% of the sucralose present in the first organic phase is extracted into the second aqueous phase by step EXT1B.

The organic extract, second organic extract 26, exits the top of extractor 22. Second organic extract 26 contains less polar impurities, such as the tetrachloro saccharides. It is purged from the process and the organic solvent recovered for reuse.

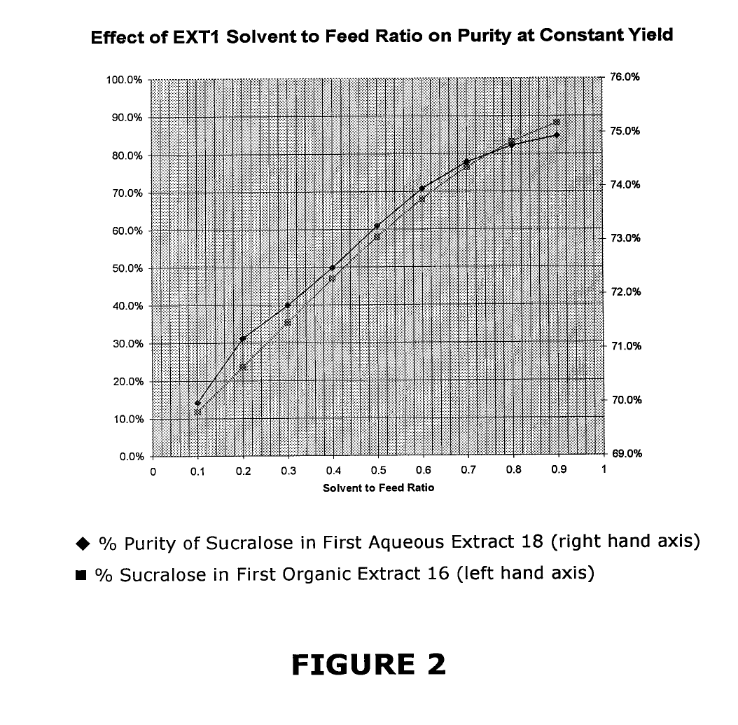

The mass ratio of organic solvent 14 to the combined aqueous feed stream in the first extraction step (EXT1) is about 0.4 to about 0.9. Preferably, the mass ratio of organic solvent 14 to aqueous feed stream 10 in step EXT1 is about 0.6 to about 0.9. FIG. 2 shows the amount of sucralose in first organic extract 16 (left hand axis) and the purity of the sucralose in first aqueous extract 18 (right hand axis) as a function of the ratio of organic solvent 14 to combined aqueous feed stream in the first extraction step (EXT1), calculated at constant sucralose yield. These values are for the process described above, a process in which a sucralose containing aqueous feed stream is extracted with an organic solvent in a first extraction step, the resulting organic extract back extracted with water in a second extraction step, and the resulting second aqueous extract recycled to the first extraction step.

As can be seen from FIG. 2, when mass ratio of organic solvent 14 to combined aqueous feed stream in the first extraction step is about 0.4 or greater, about 50% or more of the sucralose is extracted into first organic extract 16. When the mass ratio is 0.5 or greater, greater than about 60% of the sucralose is extracted into first organic extract 16. When the mass ratio is 0.6 or greater, greater than about 65% of the sucralose is extracted into first organic extract 16. Surprisingly, the level of impurities in first aqueous extract 18 is reduced significantly, with little or no decrease in overall sucralose yield, when higher organic solvent to the combined aqueous feed stream ratios are used in the first extraction step.

### Preparation of Sucrose-6-Ester

Selective protection of the 6-hydroxyl of sucrose can be carried out by reaction of sucrose with a carboxylic acid anhydride, such as acetic anhydride or benzoic anhydride, in an anhydrous polar aprotic solvent in the presence of an organotin-based acylation promoter at a temperature and for a period of time sufficient to produce the sucrose-6-ester. The 6-ester group blocks the hydroxyl on the 6 position during the chlorination reaction. Accordingly, any ester group that is stable to the conditions of the chlorination reaction and that can be removed under conditions that do not affect the resulting sucralose can be used. When sucrose-6-acetate is prepared, 1,3-diacetoxy-1,1,3,3-tetrabutyldistannoxane, for example, can be used as the organotin-based acylation promoter and acetic anhydride as the carboxylic acid anhydride. Preparation of sucrose-6-esters is disclosed in, for example, O'Brien, U.S. Pat. No. 4,783,526; Navia, U.S. Pat. No. 4,950,746; Simpson, U.S. Pat. No. 4,889,928; Neiditch, U.S. Pat. No. 5,023,329; Walkup, U.S. Pat. No. 5,089,608; Vernon, U.S. Pat. No. 5,034,551; Sankey, U.S. Pat. No. 5,470,969; Kahn, U.S. Pat. No. 5,440,026; Clark, U.S. Pat. No. 6,939,962, and Li, U.S. Pat. Pub. 2007/0227897 A1; the disclosures of which are all incorporated herein by reference.

### Conversion of Sucrose-6-Ester to Sucralose-6-Ester

To convert sucrose-6-ester to sucralose-6-ester, the hydroxyls at the 4, 1′, and 6′ positions of the sucrose-6-ester are converted to chloro groups, and the stereochemical configuration at the 4 position is inverted. Conversion of the hydroxyls in the 4, 1′, and 6′ positions of the ester to chloro groups with inversion of the stereochemical configuration at the 4 position is disclosed in Walkup, U.S. Pat. No. 4,980,463; Jai, U.S. Pat. Pub. 2006/0205936 A1; and Fry, U.S. Pat. Pub. 2007/0100139 A1; the disclosures of which are all incorporated herein by reference.

The chlorination process comprises the following steps. A reaction mixture is prepared comprising the sucrose-6-ester, a tertiary amide, and at least seven molar equivalents of a chlorination agent. For example, in one process, the sucrose-6-ester can be added in a feed stream that comprises about 20 wt % to about 40 wt % of the sucrose-6-ester. The ratio by weight of tertiary amide to total carbohydrate in the reaction mixture may be about 5:1 to about 12:1. Alternatively, a preformed chloroformiminium salt, such as (chloromethylene)dimethylammonium chloride (Arnold's reagent), can be used. (Chloromethylene)dimethylammonium chloride can be prepared, for example, by the reaction of phosgene with N,N-dimethyl formamide. Typically, the molar ratio of the (chloromethylene)dimethylammonium salt to the sucrose-6-ester is about 7:1 to about 11:1.

Subsequently, the hydroxyl groups at the 2, 3, 4, 1′, 3′, 4′, and 6′ positions of the sucrose-6-ester are converted to O-alkylformiminium groups. The resulting reaction mixture is heated at a temperature or temperatures and for a period of time or times sufficient to produce a product containing a derivative of sucralose-6-ester in which the remaining hydroxyl groups remain as O-alkylformiminium groups. For example, Walkup, U.S. Pat. No. 4,980,463, the disclosure of which is incorporated herein by reference, and Fry, U.S. 2007/0100139, the disclosure of which is incorporated herein by reference, disclose such processes.

Because formation of a chloroformiminium salt or Vilsmeier reagent is not essential to the chlorination reaction, chlorination agent refers to any compound that can be used to form a chloroformiminium salt or Vilsmeier reagent, or that can convert the hydroxyl groups of a sucrose-6-ester to chloro groups. Some chlorination agents that can be reacted with a tertiary amide to form a chloroformiminium salt include, for example, phosgene, phosphorus oxychloride, phosphorus pentachloride, thionyl chloride, sulfuryl chloride, oxalyl chloride, trichloromethyl chloroformate (“diphosgene”), bis(trichloromethyl) carbonate (“triphosgene”), and methane sulfonylchloride. Tertiary amides that can be used include, for example, N,N-dimethyl formamide (DMF), N-formyl piperidine, N-formyl morpholine, and N,N-diethyl formamide. When N,N-dimethyl formamide is used as the tertiary amide, it can also be used as the reaction solvent. Co-solvents can be used at up to about 80 vol % or more of the liquid phase of the reaction medium. Useful co-solvents are those which are both chemically inert and which provide sufficient solvent power to enable the reaction to become essentially homogeneous at the monochlorination stage, for example toluene, o-xylene, 1,1,2-trichloroethane, 1,2-diethoxyethane, diethylene glycol dimethyl ether.

Quenching of the reaction mixture restores the hydroxyl groups at the 2, 3, 3′, and 4′ positions and forms the sucralose-6-ester. The reaction mixture can be quenched by the addition of about 0.5 to about 2.0 molar equivalents, typically about 1.0 to about 1.5 molar equivalents, of alkali relative to the amount of chlorination agent used in the reaction. An aqueous solution of an alkali metal hydroxide, such as sodium or potassium hydroxide; an aqueous slurry of an alkaline earth metal hydroxide, such as calcium hydroxide; or aqueous ammonium hydroxide can be used to quench the reaction. For example, an aqueous solution of an alkali metal hydroxide, such as aqueous sodium hydroxide, that contains about 5 wt % to about 35 wt %, typically about 8 wt % to about 20 wt %, and preferably about 10 wt % to about 12 wt % can be used.

As described below, quenching can be carried out by addition of alkali to the reaction mixture, by the dual stream process, or by the circulated process. In each case pH and temperature are controlled during addition of the alkali. Quenching is typically carried out at a pH between about 8.5 to about 10.5 and at a temperature of about 0° C. to about 60° C. Preferably, the pH should not be permitted to rise above about 10.5 during the course of the quenching reaction.

In the dual stream process, quenching is carried out by slow addition of the aqueous alkali with simultaneous slow addition of the chlorination reaction material into a reaction vessel. The chlorination reaction mixture and aqueous alkali are simultaneously added slowly until the desired quantity of chlorination reaction mixture has been added. Further aqueous alkali is added until the desired pH is reached. Then the temperature and pH are maintained at the desired levels for the remainder of the reaction. This process can be a batch or continuous process.

In the circulated process, quenching is carried out by circulating the chlorination reaction mixture from a vessel through a circulation loop. Chlorination reaction mixture and aqueous alkali are added slowly into this circulation loop. Sufficient aqueous alkali is added until the desired pH is reached. Then the temperature and pH are maintained at the desired levels for the remainder of the reaction. This process can be a batch or continuous process.

Following quenching, the reaction mixture can be neutralized by the addition of aqueous acid, for example aqueous hydrochloric acid. The resulting mixture comprises sucralose 6-ester, other carbohydrate including chlorinated carbohydrate impurities, unreacted tertiary amide, and salts in an aqueous solvent in which the predominant solvent is water.

### Conversion of Sucralose-6-Ester to Sucralose

The resulting mixture typically comprises both sucralose and sucralose-6-ester. Methods for hydrolyzing sucralose-6-ester are disclosed, for example in Catani, U.S. Pat. Nos. 5,977,349, 6,943,248, 6,998,480, and 7,049,435; Vernon, U.S. Pat. No. 6,890,581; El Kabbani, U.S. Pat. Nos. 6,809,198, and 6,646,121; Navia, U.S. Pat. Nos. 5,298,611 and 5,498,709, and U.S. Pat. Pub. 2004/0030124; Liesen, U.S. Pat. Pub. 2006/0188629 A1; Fry, U.S. Pat. Pub. 2006/0276639 A1; El Kabbani, U.S. Pat. Pub. 2007/0015916 A1; Deshpande, U.S. Pat. Pub. 2007/0160732 A1; and Ratnam, U.S. Pat. Pub. 2007/0270583 A1; the disclosures of which are all incorporated herein by reference.

For example, (a) sucralose-6-ester can be hydrolyzed to sucralose by raising the pH of the reaction mixture to about 11±1 at a temperature and for a time sufficient to effect removal of the ester group, and (b) the tertiary amide removed by, for example, stream stripping. Either step (a) or step (b) can be carried first. Alternatively, conversion of sucralose-6-ester to sucralose can be carried in methanol containing sodium methoxide. A trans-esterification reaction occurs that forms sucralose and the methyl ester of the acid, for example methyl acetate when the sucralose-6-ester is sucralose-6-acetate. The methyl ester of the acid can be removed by distillation, and the resulting sucralose containing product dissolved in water.

### INDUSTRIAL APPLICABILITY

The process of the invention is useful in the preparation of sucralose. Sucralose is a high-intensity sweetener that can be used in many food and beverage applications, as well as in other applications. Such applications include, for example, beverages, combination sweeteners, consumer products, sweetener products, tablet cores (Luber, U.S. Pat. No. 6,277,409), pharmaceutical compositions (Luber, U.S. Pat. No. 6,258,381; Roche, U.S. Pat. No. 5,817,340; and McNally, U.S. Pat. No. 5,593,696), rapidly absorbed liquid compositions (Gelotte, U.S. Pat. No. 6,211,246), stable foam compositions (Gowan, Jr., U.S. Pat. No. 6,090,401), dental floss (Ochs, U.S. Pat. No. 6,080,481), rapidly disintegrating pharmaceutical dosage forms (Gowan, Jr., U.S. Pat. No. 5,876,759), beverage concentrates for medicinal purposes (Shah, U.S. Pat. No. 5,674,522), aqueous pharmaceutical suspensions (Ratnaraj, U.S. Pat. No. 5,658,919; Gowan, Jr. U.S. Pat. Nos. 5,621,005 and 5,374,659; and Blase, U.S. Pat. Nos. 5,409,907 and 5,272,137), fruit spreads (Antenucci, U.S. Pat. No. 5,397,588; and Sharp, 5,270,071), liquid concentrate compositions (Antenucci, U.S. Pat. No. 5,384,311), and stabilized sorbic acid solutions (Merciadez, U.S. Pat. No. 5,354,902). The determination of an acceptable sweetness can be accomplished by a variety of standard “taste test” protocols known in the art which are well known to those skilled in the art, such as, for example, the protocols referred to in Merkel, U.S. Pat. No. 6,998,144, and Shamil, U.S. Pat. No. 6,265,012.

The advantageous properties of this invention can be observed by reference to the following example which illustrates but does not limit the invention.

### EXAMPLES

### Example 1

This example was generated using a mathematical model that included both a first extraction process (EXT1), a back extraction (EXT1B) of the first organic extract (16), and recycle of the second aqueous extract (12) to the first extraction process. The calculations used in the model were derived from theoretical equations fitted to actual pilot plant data. FIG. 1 shows a flow diagram of the modeled process.

FIG. 2 shows the results from multiple model runs in which the mass ratio of organic solvent 14 to the combined aqueous feed stream in the first extraction was varied. The number of separation stages in the back extraction was adjusted to maintain an equivalent overall extraction yield. The amount of sucralose extracted into the first organic extract 16 during the first extraction step is shown on the left hand axis. The purity of the sucralose produced by the process is shown on the right hand axis.

As can be seen from FIG. 2, when the mass ratio of organic solvent 14 to combined aqueous feed stream in the first extraction step is about 0.4 or greater, about 50% or more of the sucralose is extracted into first organic extract 16. When the mass ratio is 0.6 or greater, greater than about 65% of the sucralose is extracted into first organic extract 16. Surprisingly, the level of impurities in first aqueous extract 18 is reduced significantly, with little or no reduction in overall sucralose yield, when higher organic solvent to the combined aqueous feed stream ratios are used in the first extraction step. As can also be seen from FIG. 2, product purity begins to level out near 75%, when nearly 90% of the sucralose is extracted into first organic extract 16.

### Example 2

To further illustrate the utility of the invention, partition coefficients of ethyl acetate, isopropyl acetate, n-propyl acetate, n-butyl acetate, methyl isobutyl ketone, and n-butanol were measured. The partition coefficients were measured between an aqueous crude (unpurified) sucralose solution prepared as set out above by acetylation, chlorination, and deacetylation of sucrose, and each of the solvents. The partition coefficients were entered into the same mathematical model as used in Example 1, and new simulations were completed. The purity of the starting material simulated in the model was 63% by weight.

The following procedure was used to generate the data. First, for the EXT1 extraction, a solvent to feed ratio and total carbohydrate concentration in the aqueous feed were selected so that >50% of the sucralose and >95% of the tetrachloro saccharide impurities were extracted into the solvent phase. Parameters for the EXT1B unit were then adjusted so that the chemical yield in the purification process was the same for each of the solvents modeled: the parameters that were variable were the number of stages and the solvent:water ratio. The purity of sucralose in the aqueous stream from the EXT1 extraction was then determined each case according to the mathematical model.

Table 1 below shows the results from all the model runs. Results similar to those obtained for ethyl acetate could be obtained for all of the solvents. In most cases, the purity of sucralose in the first aqueous extract 18 was close to the optimal value established in Example 1.

TABLE 1

| | % | Sucralose | | | | | |
|---|---|---|---|---|---|---|---|
| Solvent | Sucralose | Purity in | | | | | Total |
| to Feed | in EXT1 | EXT1 | | | | EXT1B | Carb |
| Ratio in | Organic | aqueous | Overall | EXT1B | Solvent | solvent:water | Conc in |
| EXT1 | Extract 16 | stream 18 | Yield | Stages | Used | ratio | feed |
| 0.7 | 73.3% | 74.8% | 96.0% | 41 | Ethyl | 0.8 | 10 |
| | | | | | Acetate | | |
| 0.6 | 67.9% | 74.4% | 96.0% | 20.0 | Isopropyl | 0.70 | 20 |
| | | | | | Acetate | | |
| 0.7 | 67.3% | 74.8% | 96.0% | 14.5 | n-Propyl | 0.75 | 20 |
| | | | | | Acetate | | |
| 0.7 | 75.1% | 74.8% | 96.0% | 27.0 | Methyl | 0.75 | 18 |
| | | | | | Isobutyl | | |
| | | | | | Ketone | | |
| 0.2 | 73.7% | 70.7% | 96.0% | 22.0 | n-Butanol | 0.21 | 5.4 |

The disclosure of the invention includes the following claims. Having described the invention, we now claim the following and their equivalents.

What is claimed is:

1. A process for the purification of sucralose, the process comprising the steps of:
a) extracting an aqueous stream comprising sucralose and tetrachloro saccharides with an organic solvent and producing a first organic extract and a first aqueous extract, in which the organic solvent is immiscible with water, and in which greater than 50% of the sucralose and at least 95% of the tetrachloro saccharides in the aqueous stream pass into the first organic extract;
b) extracting the first organic extract with an aqueous solvent to produce a second organic extract and a second aqueous extract, in which the sucralose preferentially passes into the second aqueous extract; and
c) adding the second aqueous extract to step a).

2. The process of claim 1 in which the organic solvent comprises a solvent selected from the group consisting of methyl acetate, ethyl acetate, iso-propyl acetate, n-propyl acetate, n-butyl acetate, amyl acetate, methyl ethyl ketone, methyl iso-butyl ketone, methyl iso-amyl ketone, methylene chloride, chloroform, n-butanol, and mixtures thereof; preferably comprises a solvent selected from the group consisting of ethyl acetate, iso-propyl acetate, n-propyl acetate, n-butyl acetate, methyl iso-butyl ketone, n-butanol, and mixtures thereof, and more preferably comprises ethyl acetate.

3. The process of claim 2 in which the organic solvent is ethyl acetate.

4. The process of claim 1 in which the mass ratio of the organic solvent to the aqueous feed stream is about 0.4 to about 0.9.

5. The process of claim 1 in which the mass ratio of the organic solvent to the aqueous feed stream is about 0.5 to about 0.9.

6. The process of claim 1 in which the mass ratio of the organic solvent to the aqueous feed stream is about 0.6 to about 0.9.

7. The process of claim 1 in which, in step a), greater than 55% of the sucralose in the aqueous stream passes into the first organic extract.

8. The process of claim 1 in which, in step a), greater than 60% of the sucralose in the aqueous stream passes into the first organic extract.

9. The process of claim 1 in which, in step a), greater than 65% of the sucralose in the aqueous stream passes into the first organic extract.

10. The process of claim 1 in which, in step b), greater than 90% of the sucralose in the first organic extract passes into the second aqueous extract.

11. The process of claim 1 in which, in step b), greater than 92% of the sucralose in the first organic extract passes into the second aqueous extract.

12. The process of claim 1 in which, in step b), greater than 95% of the sucralose in the first organic extract passes into the second aqueous extract.

13. The process of claim 1 additionally comprising the steps of concentrating the first aqueous extract to form a concentrated extract and crystallizing sucralose from the concentrated extract.

14. The process of claim 1 additionally comprising the step or steps of purifying and isolating the sucralose.
